(19) United States
(12) Patent Application Publication
(10) Pub. No.: US 2026/0047888 A1
(43) Pub. Date: Feb. 19, 2026

(54) MIXED REALITY BONE GRAFT SHAPING

(71) Applicant: Howmedica Osteonics Corp., Mahwah, NJ (US)

(72) Inventors: Benjamin Dassonville, Saint Hilaire du Touvet (FR); Nicolas Neichel, Le Sappey En Chartreuse (FR); Vincent Gaborit, Saint Martin D’Hères (FR)

(21) Appl. No.: 19/104,664

(22) PCT Filed: Sep. 7, 2023
(86) PCT No.: PCT/US2023/032199

§ 371 Date: Feb. 18, 2025

Related U.S. Application Data
(60) Provisional application No. 63/375,151, filed on Sep. 9, 2022.

(51) Int. Cl. A61B 34/10; A61B 17/15; A61B 17/16; A61B 17/17; A61B 34/00; A61B 34/20; A61B 90/00; A61B 90/50

(52) U.S. Cl. CPC A61B 34/10; A61B 17/15; A61B 17/1604; A61B 17/1635; A61B 17/1778; A61B 34/20; A61B 34/25; A61B 90/37; A61B 90/39; A61B 90/50; A61B 2034/102; A61B 2034/105; A61B 2034/2048; A61B 2034/2051; A61B 2034/2055; A61B 2034/2065; A61B 2090/365; A61B 2090/372; A61B 2090/3983; A61B 2090/502

(57) ABSTRACT

Techniques and systems are described for mixed reality bone graft cutting. A method comprises, receiving tracking input of a scene in which a target bone is positioned on a bone support member of a platform, wherein the platform that comprises a reference marker; generating registration data that registers the reference marker with a coordinate system; obtaining data defining a planned surface of die target bone; determining, based on the registration data, a position in the coordinate system for a virtual object representing the planned surface of the target bone; and while the target bone is positioned on the bone support member of the platform, causing a mixed reality (MR) visualization device to output the virtual object for display so that the virtual object appears to a user of the MR visualization device to pass through the target bone and indicates the planned surface of the target bone.

This application claims the benefit of priority to U.S. Provisional Patent Application 63/375,151, filed Sep. 9, 2022, the entire content of which is incorporated by reference.

### BACKGROUND

Some orthopedic surgeries involve attaching a bone fragment between a patient's natural bone and an orthopedic prosthesis. For example, in a shoulder arthroplasty, a surgeon may insert a bone fragment (i.e., a bone graft) between the glenoid fossa of a patient's scapula and a glenoid prosthesis. Insertion of the bone graft may help stabilize the glenoid prosthesis and avoid further damage to the patient's scapula, which may be especially important in cases where the scapula has experienced significant erosion or trauma.

### SUMMARY

This disclosure describes techniques for providing mixed reality (MR) guidance for shaping bone grafts. As described herein, a computing system may receive tracking input of a scene in which a target bone is positioned on a bone support member of a platform. The platform comprises a reference marker. The computing system may generate registration data that registers at least one of the target bone, the bone support member, or the platform with a coordinate system based on the reference marker. Additionally, the computing system may obtain data defining a planned surface of the target bone. While the target bone is positioned on the bone support member of the platform and while a position in the coordinate system of the target bone is within a field of view of a MR visualization device, the MR visualization device may output a virtual object for display so that the virtual object appears to a user of the MR visualization device to pass through the target bone and indicates the planned surface of the target bone. That is, the virtual object may appear to the user of the MR visualization device to pass through the target bone, and the virtual object indicates the planned surface of the target bone based on appearing to pass through the target bone.

In one example, this disclosure describes a method comprising: receiving, by a computing system, tracking input of a scene in which a target bone is positioned on a bone support member of a platform, wherein the platform comprises a reference marker; generating, by the computing system, registration data that registers the reference marker with a coordinate system; obtaining, by the computing system, data defining a planned surface of the target bone; determining, by the computing system, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of a mixed reality (MR) visualization device, causing, by the computing system, the MR visualization device to output the virtual object for display so that the virtual object appears to a user of the MR visualization device to pass through the target bone and indicates the planned surface of the target bone.

In another example, this disclosure describes a system comprising: a mixed reality (MR) visualization device; and processing circuitry configured to: receive tracking input of a scene in which a target bone is positioned on a bone support member of a platform, wherein the platform comprises a reference marker; generate registration data that registers the reference marker with a coordinate system; obtain data defining a planned surface of the target bone; determine, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of the MR visualization device, cause the MR visualization device to output the virtual object for display so that the virtual object appears to a user of the MR visualization device to pass through the target bone and indicates the planned surface of the target bone.

In another example, this disclosure describes a non-transitory computer-readable storage medium having instructions stored thereon that, when executed, cause a computing system to receive tracking input of a scene in which a target bone is positioned on a bone support member of a platform, wherein the platform comprises a reference marker; generate registration data that registers the reference marker with a coordinate system; obtain data defining a planned surface of the target bone; determine, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of a mixed reality (MR) visualization device, cause the MR visualization device to output the virtual object for display so that the virtual object appears to a user of the MR visualization device to pass through the target bone and indicates the planned surface of the target bone.

In another example, this disclosure describes a system comprising: means for receiving tracking input of a scene in which a target bone is positioned on a bone support member of a platform, wherein the platform comprises a reference marker; means for generating registration data that registers the reference marker with a coordinate system; means for obtaining data defining a planned surface of the target bone; determining, by the computing system, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of a mixed reality (MR) visualization device, causing, by the computing system, the MR visualization device to output the virtual object for display so that the virtual object appears to a user of the MR visualization device to pass through the target bone and indicates the planned surface of the target bone.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a conceptual diagram illustrating an example system in which one or more techniques of this disclosure may be performed.

FIGS. 2A-2F are conceptual diagrams illustrating an example process of preparing a bone graft according to techniques of this disclosure.

FIG. 3 is a conceptual diagram illustrating an example of placing a target bone on a platform according to techniques of this disclosure.

FIG. 4 is a conceptual diagram illustrating bone fragment removal according to techniques of this disclosure.

FIG. 5 is a conceptual diagram illustrating a virtual object indicating a complex planned surface of a bone according to techniques of this disclosure.

FIG. 6 is a conceptual diagram illustrating a bone having a modified surface according to techniques of this disclosure.

FIG. 7 is a block diagram illustrating an example computing system in accordance with one or more techniques of this disclosure.

FIG. 8 is a schematic representation of a mixed reality (MR) visualization device in accordance with one or more techniques of this disclosure.

FIG. 9 is a conceptual diagram illustrating an example user interface of a planning system for a bone graft, according to techniques of this disclosure.

FIG. 10 is a conceptual diagram illustrating an example user interface providing details of a planned bone graft, according to techniques of this disclosure.

FIG. 11 is a flowchart illustrating an example operation of a computing system in accordance with one or more techniques of this disclosure,

FIG. 12 is a conceptual diagram illustrating an example system in which a clinician uses robotic assistance to shape a target bone in accordance with one or more techniques of this disclosure.

### DETAILED DESCRIPTION

A surgeon may implant an orthopedic prosthesis into a patient during a surgery. For example, during a shoulder arthroplasty surgery, a surgeon may implant a glenoid prosthesis onto a patient's scapula. To achieve the desired position and orientation of the orthopedic prosthesis, the surgeon may place a bone graft between the orthopedic prosthesis and the patient's existing bone. The bone graft is a bone fragment that has been removed from another bone of the patient or a donor. The bone graft may form a spacer between the orthopedic prosthesis and the patient's existing bone. The surgeon may attach the orthopedic prosthesis to the bone graft and the patient's existing bone by driving screws or other types of fixation devices through a base plate of the orthopedic prosthesis, the bone graft, and into the patient's existing bone. Because the bone graft is a piece of bone, the patient's body may naturally form a strong bond between the bone graft and the patient's existing bone.

For a bone graft to function properly as a spacer between an orthopedic prosthesis and the patient's existing bone, the bone graft may need to have a shape that is specific to the patient's anatomy. For example, the patient's glenoid fossa may have a complex and irregular shape, especially if there has been significant bone erosion or trauma. Shaping the bone graft to a shape specific to the patient's anatomy may ensure that there is strong and stable contact between the patient's natural bone and the bone graft, which allows for a strong bond to form between the patient's natural bone and the bone graft.

During a shoulder arthroplasty surgery, the surgeon may remove a cylindrical section of bone from the humeral head of the patient's humerus to serve as a bone graft. The humeral head may be a good source for harvesting the bone graft because the humeral head is typically removed during a total shoulder arthroplasty and replaced with a humeral prosthesis. After removing the bone graft from the humeral head, the surgeon may shape the bone graft so that the bone graft is shaped to fit between bone of the patient's glenoid fossa and a base plate of an orthopedic prosthesis so that the orthopedic prosthesis is at a planned position and orientation. The surgeon may use cutting tools such as an oscillating saw or cutting burr to shape the bone graft. Shaping the bone graft so that the bone graft has the correct shape under the time pressures of surgery may be challenging, especially when the correct shape is complex.

This disclosure describes techniques for mixed reality (MR) bone graft shaping. As described herein, a computing system receives tracking input of a scene in which a target bone is positioned on a bone support member of a platform. The platform also comprises a reference marker. The computing system may generate registration data that registers the reference marker with a coordinate system. Furthermore, the computing system may obtain data defining a planned surface. The planned surface may be defined in a surgical plan for shaping the bone for use as a bone graft. While the target bone is positioned on the bone support member of the platform and while a position in the coordinate system of the target bone is within a field of view of a MR visualization device, an MR visualization device may output a virtual object for display so that the virtual object appears to a user of the MR visualization device to pass through the target bone and indicates the planned surface of the target bone.

Applying the techniques of this disclosure may enable a clinician to shape the bone for use as a bone graft quicker and with greater precision. Because the platform comprises the reference marker, e.g., as opposed to the target bone itself, the reference marker may be more visible and more stable than if attached to the bone. For instance, the clinician's hands are less likely to obscure the reference marker from view of sensors in the MR visualization device. Hence, there is a lower likelihood of losing registration between the target bone and the virtual object. Moreover, inclusion of the reference marker on the platform may have greater accuracy than tracking the target bone with markerless registration because changes to the shape of the target bone may make markerless tracking difficult. Additionally, because the virtual object may be overlaid on the target bone, it may not be necessary for the clinician to look away from the target bone to a separate monitor to see the planned surface with respect to the target bone. This may improve the accuracy and speed of the clinician when shaping the target bone.

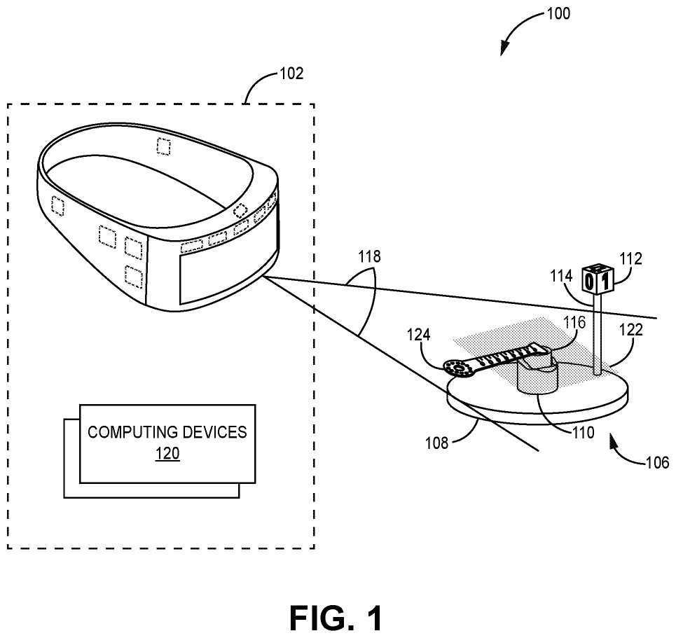

FIG. 1 is a conceptual diagram illustrating an example system 100 in which one or more techniques of this disclosure may be performed. In the example of FIG. 1, system 100 includes a computing system 102, which includes a MR visualization device 104. System 100 also includes a platform 106. In the example of FIG. 1, platform 106 includes a base plate 108, a bone support member 110, a reference marker 112, and a marker stem 114. A target bone 116 may be placed in bone support member 110 during a process of shaping target bone 116 for use as a bone graft. A clinician may wear MR visualization device 104. The clinician may be a surgeon, nurse, technician, medic, physician, or other type of medical professional or person. In the example of FIG. 1, base plate 108 is circular, but in other examples, base plate 108 may have other shapes.

In the example of FIG. 1, target bone 116 is initially cylindrical. Bone support member 110 is also cylindrical and has a raised rim having an inner diameter that substantially matches an outer diameter of target bone 116. Thus, the inner diameter of the raised rim of bone support member 110 may hold target bone 116 securely in position while target bone 116 is being reshaped.

In the example of FIG. 1, reference marker 112 is an optical marker having predefined optical patterns on different faces of a cube. For instance, reference marker 112 may be a cube having different predefined optical patterns on each face other than a face to which marker stem 114 is connected. In the example of FIG. 1, reference marker 112 has numbers on different faces. In other examples, the faces of reference marker 112 may have 2-dimensional optical codes, such as Quick Response (QR) codes or other types of matrix barcodes. Furthermore, in other examples, the faces of reference marker 112 have different predefined optical patterns. In other examples, reference marker 112 has different shapes. For instance, in other examples, reference marker 112 may be a dodecahedron, pyramid, or another shape. In some examples, reference marker 112 may be an ultrasonic emitter, an electromagnetic marker, a passive optical marker that reflects light, an active optical marker that emits light, and so on. In some examples, reference marker 112 comprises a set of objects (e.g., balls, cubes, etc.) having predefined sizes and arranged in a predefined spatial configuration.

Reference marker 112 may be at a predefined position relative to bone support member 110. For instance, reference marker 112 may be at a predefined radius and/or elevation relative to bone support member 110. As described in detail elsewhere in this disclosure, the fact that reference marker 112 is at a predefined position relative to bone support member 110 may enable MR visualization device 104 to determine a position and orientation of MR visualization device 104 relative to bone support member 110, and hence target bone 116, more efficiently and accurately.

In the example of FIG. 1, marker stem 114 supports reference marker 112. Marker stem 114 may be at a predefined radial distance from bone support member 110. For example, marker stem 114 may be 10 centimeters (cm), 15 cm, or another predefined distance from bone support member 110. Furthermore, marker stem 114 may support reference marker 112 at a predefined elevation above base plate 108. In other examples, reference marker 112 may be included directly in base plate 108 or otherwise retained at a predefined position relative to bone support member. However, raising reference marker 112 to an elevation above an anticipated elevation of a top of target bones positioned in bone support member 110 may allow reference marker 112 to be in view of optical sensors of MR visualization device 104 from a wider variety of positions, including when the clinician is looking at target bone 116 from an angle parallel to base plate 108. In the example of FIG. 1, lines 118 correspond to a field of view of the clinician while wearing MR visualization device 104.

MR visualization device 104 may use various visualization techniques to display MR visualizations to the clinician. A MR visualization may comprise one or more virtual objects that are viewable by a user at the same time as real-world objects. Thus, what the clinician sees may be a mixture of real and virtual objects.

MR visualization device 104 may comprise various types of devices for presenting MR visualizations. For instance, in some examples, MR visualization device 104 may be a Microsoft HOLOLENS™ headset, such as the HOLOLENS 2 headset, available from Microsoft Corporation, of Redmond, Washington, USA, or a similar device, such as, for example, a MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, MR visualization device 104 may be a holographic projector, head-mounted smartphone, special-purpose MR visualization device, or another type of device for presenting MR visualizations. In some examples, MR visualization device 104 includes a head-mounted unit and a backpack unit that performs at least some of the processing functionality of MR visualization device 104. In other examples, all functionality of MR visualization device 104 is performed by hardware residing in a head-mounted unit. Discussion in this disclosure of actions performed by system 100 may be performed computing system 102 of system 100, MR visualization device 104, or a combination of the one or more computing devices and MR visualization device 104.

In the example of FIG. 1, computing system 102 may comprise one or more computing devices 120 in addition to MR visualization device 104. Computing devices 120 may include server computers, personal computers, smartphones, tablet computers, laptop computers, and other types of computing devices. Computing devices 120 may perform at least some of the computing tasks of computing system 102. In some examples, MR visualization device 104 may be one or the only computing device of computing system 102. In examples where computing system 102 includes computing devices 120 in addition to MR visualization device 104, computing devices 120 may communicate with MR visualization device 104 via one or more wired or wireless communication links.

While target bone 116 is positioned in bone support member 110 and while the target bone is within a field of view of MR visualization device 104, MR visualization device 104 may output a virtual object 122 for display so that virtual object 122 appears to the clinician to pass through target bone 116 and indicates a planned surface of target bone 116. In the example of FIG. 1, virtual object 122 indicates a cutting plane (i.e., a plane along which the clinician is to cut target bone 116) along the planned surface of target bone 116. In other words, a saw blade 124 of an oscillating saw (not shown) may be used to cut target bone 116 along a surface indicated by virtual object 122. MR visualization device 104 may continue to output virtual object 122 for display while the clinician is using a tool (e.g., saw blade 124) to shape target bone 116. Although the example of FIG. 1 shows virtual object 122 as a plane, virtual object 122 may have more complex shapes. For instance, virtual object 122 may represent a curve, a composition of two or more planes or curves, and so on.

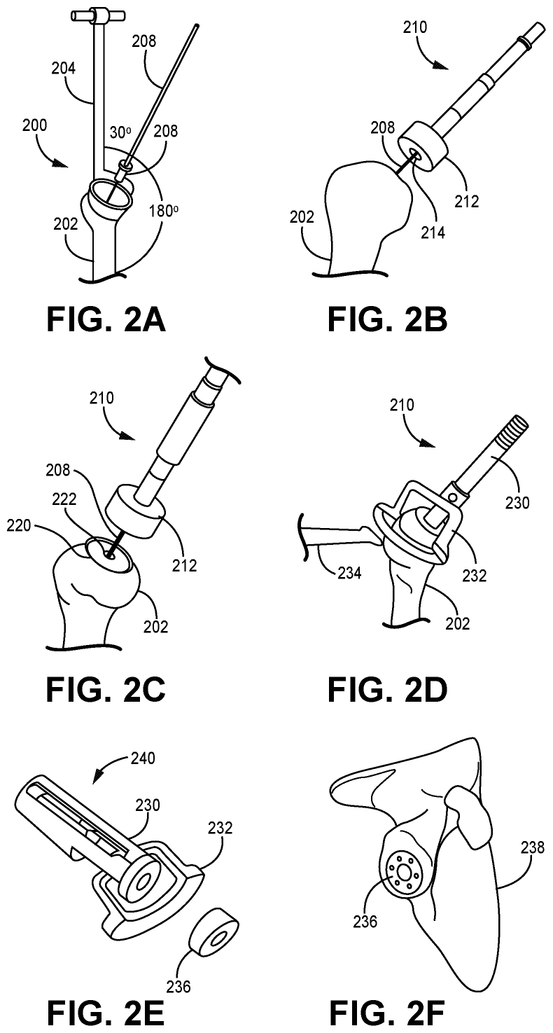

FIGS. 2A-2F are conceptual diagrams illustrating an example process of preparing a bone graft according to techniques of this disclosure. FIG. 2A shows a pin guidance jig 200 attached to a humerus 202. Pin guide jig 200 includes a punch element 204 that penetrates humerus 202 along a lengthwise axis of humerus 202. Pin guidance jig 200 also includes a cannulated element 206. Cannulated element 206 defines a channel that guides a surgical pin 208 into a head of humerus 202. In the example of FIG. 2A, there is a 145° angle between the lengthwise axis of humerus 202 and an axis through the channel defined by cannulated element 206 and there is a 35° angle between the lengthwise axis of punch element 204 and the axis passing through the channel defined by cannulated element 206. After surgical pin 208 has been inserted into the head of humerus 202, pin guide jig 200 may be removed.

FIG. 2B shows an annular cutting element 210. Compound cutting element 210 includes an outer cutting element 212 and a central cutting element 214. Central cutting element 214 is cannulated so that surgical pin 208 may pass through a channel defined in central element 214. In this way, surgical pin 208 retains compound cutting element 210 at a planned position and orientation relative to humerus 202. When compound cutting element 210 is used, outer cutting element 212 cuts a circular incision in humerus 202. In addition, central cutting element 214 cuts a smaller circular incision surrounding surgical pin 208.

FIG. 2C shows compound cutting element 210 and humerus 202 after compound cutting element 210 has been used on humerus 202. A circular incision 220 in humerus 202 made by outer cutting element 212 and a circular incision 222 in humerus 202 made by central cutting element 214 are visible in part 3 of FIG. 2. Surgical pin 208 remains inserted in humerus 202 after the incisions are made in humerus 202.

FIG. 2D shows saw guide 240 attached to humerus 202. Saw guide 240 includes a cannulated element 230 and a cutting plane element 232 connected to cannulated element 230. Cannulated element 230 defines a channel that accommodates surgical pin 208. A clinician may insert a saw blade 234 of an oscillating saw into humerus along a distal surface of cutting plane element 232. In this way, saw blade 234 severs a portion of humerus 202 along the distal surface of cutting plane element 232. A plane defined by cutting plane element 232 intersects circular incision 220 and circular incision 222. Thus, as shown in FIG. 2E, a cylindrical bone fragment 236 is extracted from humerus 202. As shown in FIG. 2F, after a clinician shapes bone fragment 236 using techniques of this disclosure, bone fragment 236 may be used as a bone graft attached to a scapula 238 of a patient.

Although the example of FIG. 2F shows the bone graft being attached to a scapula, the techniques of this disclosure may be applicable with respect to other parts of the body of a patient, such as a foot, ankle, knee, hip, elbow, spine, wrist, hand, jaw, cranium, ribs, chest, and so on.

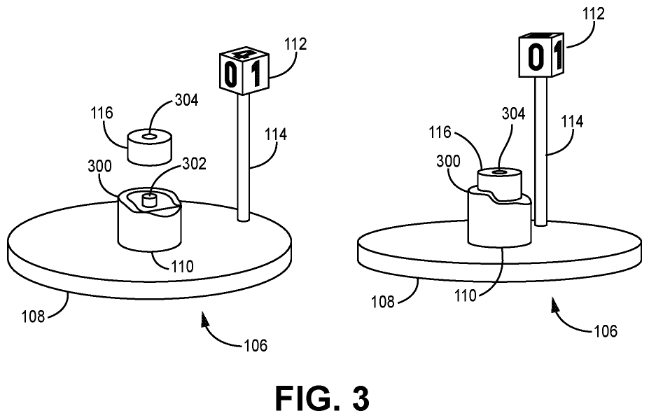

FIG. 3 is a conceptual diagram illustrating an example of placing a target bone 116 on a platform according to techniques of this disclosure. Target bone 116 may be bone fragment 236 of FIG. 2. In other examples, target bone 116 may be obtained in ways that differ from the example of FIG. 2. As shown in the example of FIG. 3, bone support member 110 has a raised rim 300 and a raised central protrusion 302. Rim 300 has an inner diameter that approximately matches an outer diameter of target bone 116. Central protrusion 302 has a diameter that approximately matches a diameter of a central circular incision 304 in target bone 116. In this way, rim 300 and central protrusion 302 may serve to retain target bone 116 at a stable position relative to platform 106 (and therefore at a stable position relative to reference marker 112).

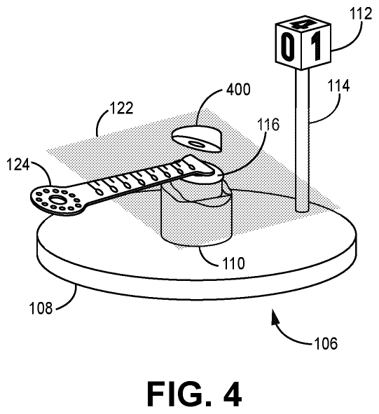

FIG. 4 is a conceptual diagram illustrating bone fragment removal according to techniques of this disclosure. As mentioned above, MR visualization device 104 may output virtual object 122 for display. Virtual object 122 may guide the clinician to use saw blade 124 to cut target bone 116 along a cutting plane. In this way, the clinician may remove bone fragment 400. In some examples, target bone 116 is used as a bone graft in an orthopedic surgery. In some examples, bone fragment 400 is used as a bone graft in an orthopedic surgery.

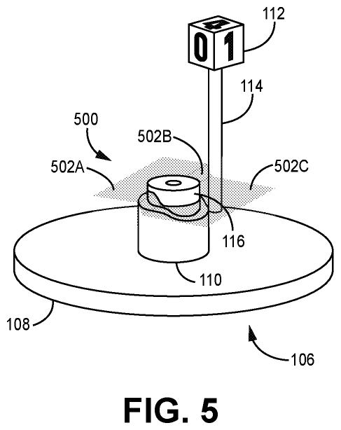

FIG. 5 is a conceptual diagram illustrating a virtual object 500 indicating a complex planned surface of target bone 116 according to techniques of this disclosure. In the example of FIG. 5, the planned surface of target bone 116 shown by virtual object 500 comprises multiple planes 502A, 502B, 502C (collectively, “planes 502”). Planes 502 may be selected by a clinician or computerized planning system so that a bone fragment (e.g., target bone 116 or a fragment of target bone 116) conforms to a shape of bone to which the bone fragment will be grafted. In other examples, virtual object 500 may include more or fewer planes, include curves, and so on.

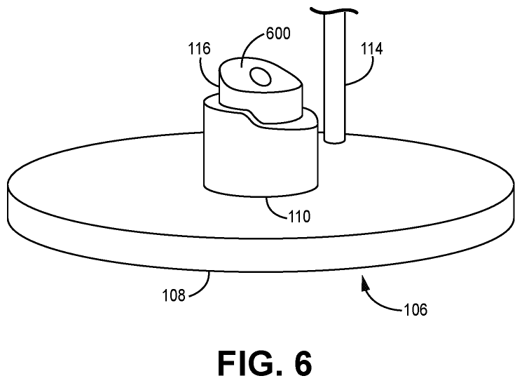

FIG. 6 is a conceptual diagram illustrating target bone 116 having a modified surface 600 according to techniques of this disclosure. As shown in the example of FIG. 6, modified surface 600 has a complex shape comprising two or more planes. A clinician may shape target bone 116 to have modified surface 600 with assistance of virtual guidance presented according to techniques of this disclosure. For instance, a surgeon may shape target bone 116 to have modified surface 600 by cutting target bone 116 according to planes 502 shown in FIG. 5.

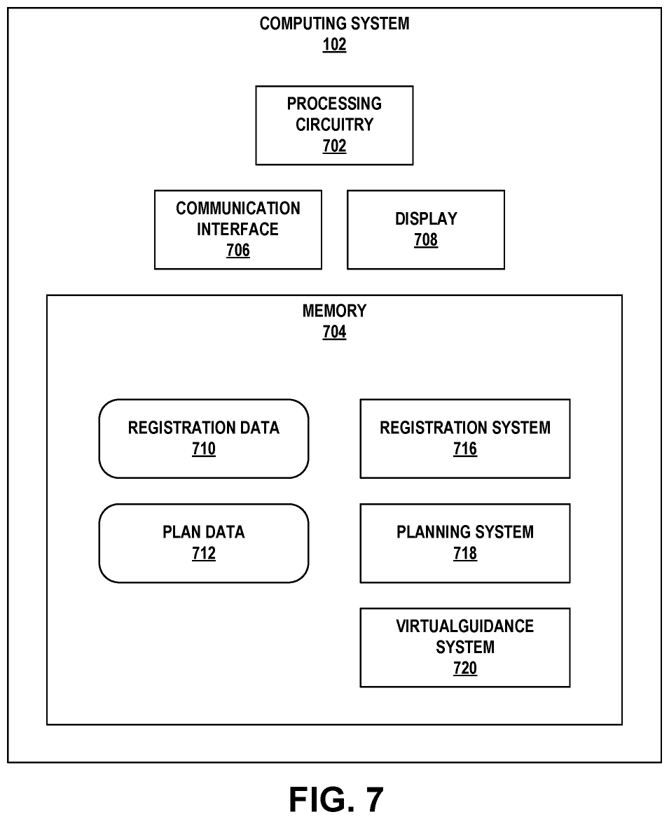

FIG. 7 is a block diagram illustrating an example computing system 102 in accordance with one or more techniques of this disclosure. In the example of FIG. 7, computing system 102 includes processing circuitry 702, memory 704, a communication interface 706, and a display 708. In other examples, computing system 102 may include more, fewer, or different components. The components of computing system 102 may be in one or more computing devices. For example, processing circuitry 702 may be in a single computing device or distributed among multiple computing devices of computing system 102, memory 704 may be in a single computing device or distributed among multiple computing devices of computing system 102, and so on.

Examples of processing circuitry 702 include one or more microprocessors, digital signal processors (DSPs), application specific integrated circuits (ASICs), field programmable gate arrays (FPGAs), hardware, or any combinations thereof. In general, processing circuitry 702 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, one or more of the units may be distinct circuit blocks (fixed-function or programmable), and in some examples, the one or more units may be integrated circuits.

Processing circuitry 702 may include arithmetic logic units (ALUs), elementary function units (EFUs), digital circuits, analog circuits, and/or programmable cores, formed from programmable circuits. In examples where the operations of processing circuitry 702 are performed using software executed by the programmable circuits, memory 704 may store the object code of the software that processing circuitry 702 receives and executes, or another memory within processing circuitry 702 (not shown) may store such instructions. Examples of the software include software designed for surgical planning. Processing circuitry 702 may perform the actions ascribed in this disclosure to computing system 102.

Memory 704 may store various types of data used by processing circuitry 702. Memory 704 may include any of a variety of memory devices, such as dynamic random access memory (DRAM), including synchronous DRAM (SDRAM), magnetoresistive RAM (MRAM), resistive RAM (RRAM), or other types of memory devices. Examples of display 708 include a liquid crystal display (LCD), a plasma display, an organic light emitting diode (OLED) display, or another type of display device.

Communication interface 706 that allows computing system 102 to output data and instructions to and receive data and instructions from MR visualization device 104, a medical imaging system, or other device via one or more communication links or networks. Communication interface 706 may be hardware circuitry that enables computing system 102 to communicate (e.g., wirelessly or using wires) to other computing systems and devices, such as MR visualization device 104. Example networks may include various types of communication networks including one or more wide-area networks, such as the Internet, local area networks, and so on. In some examples, the network may include wired and/or wireless communication links.

In the example of FIG. 7, memory 704 stores registration data 710 and plan data 712. Additionally, in the example of FIG. 7, memory 704 stores a registration system 716, a planning system 718, and virtual guidance system 720. In other examples, memory 704 may store more, fewer, or different types of data or units. Moreover, the data and units illustrated in the example of FIG. 7 are provided for purposes of explanation and may not represent how data is actually stored or how software is actually implemented. Registration system 716, planning system 718, and virtual guidance system 720 may comprise instructions that are executable by processing circuitry 702. For ease of explanation, this disclosure may describe registration system 716, planning system 718, and virtual guidance system 720 as performing various actions when processing circuitry 702 executes instructions of registration system 716, planning system 718, and virtual guidance system 720.

Computing system 102 may receive tracking input of a scene in which target bone 116 is positioned on a bone support member 110 of a platform 106 that comprises reference marker 112. Registration system 716 may generate registration data that registers reference marker 112 with a coordinate system. The registration data may define transforms between a virtual coordinate system and the “real world” coordinate system.

As part of performing the registration process, registration system 716 may generate a first point cloud and a second point cloud. The first point cloud includes points corresponding to landmarks on one or more virtual objects, such as a virtual object representing a planned surface of a target bone. The second point cloud includes points corresponding to landmarks on real-world objects, such as reference marker 112. Landmarks may be locations on virtual or real-world objects. The points in the first point cloud may be expressed in terms of coordinates in a first coordinate system and the points in the second point cloud may be expressed in terms of coordinates in a second coordinate system. Because virtual objects may be designed with positions that are relative to one another but not relative to any real-world objects, the first and second coordinate systems may be different.

Registration system 716 may generate the second point cloud using a Simultaneous Localization and Mapping (SLAM) algorithm. By performing the SLAM algorithm, registration system 716 may generate the second point cloud based on the tracking data. Registration system 716 may perform one of various implementations of SLAM algorithms, such as a SLAM algorithm having a particular filter implementation, an extended Kalman filter implementation, a covariance intersection implementation, a GraphSLAM implementation, an ORB SLAM implementation, or another implementation. In some examples, registration system 716 applies an outlier removal process to remove outlying points in the first and/or second point clouds. In some examples, the outlying points may be points lying beyond a certain standard deviation threshold from other points in the point clouds. Applying outlier removal may improve the accuracy of the registration process.

In some examples, as part of performing the registration process, registration system 716 may apply an image recognition process that uses the tracking data to identify reference marker 112. Identifying reference marker 112 may enable registration system 716 to determine a preliminary spatial relationship between points in the first point cloud and points in the second point cloud. The preliminary spatial relationship may be expressed in terms of translational and rotational parameters.

Next, registration system 716 may refine the preliminary spatial relationship between points in the first point cloud and points in the second point cloud. For example, registration system 716 may perform an iterative closest point (ICP) algorithm to refine the preliminary spatial relationship between the points in the first point cloud and the points in the second point cloud. The iterative closest point algorithm finds a combination of translational and rotational parameters that minimize the sum of distances between corresponding points in the first and second point clouds. For example, consider a basic example where landmarks corresponding to points in the first point cloud are at coordinates A, B, and C and the same landmarks correspond to points in the second point cloud are at coordinates A′, B′, and C′. In this example, the iterative closest point algorithm determines a combination of translational and rotational parameters that minimizes ΔA+ΔB+ΔC, where ΔA is the distance between A and A′, ΔB is the distance between B and B′, and ΔC is the distance between C and C′. To minimize the sum of distances between corresponding landmarks in the first and second point clouds, registration system 716 may perform the following steps:
1. For each point of the first point cloud, determine a corresponding point in the second point cloud. The corresponding point may be the closest point in the second point cloud. In this example, the first point cloud includes points corresponding to landmarks on one or more virtual objects and the second point cloud may include points corresponding to landmarks on real-world objects (e.g., reference marker 112).
2. Estimate a combination of rotation and translation parameters using a root mean square point-to-point distance metric minimization technique that best aligns each point of the first point cloud to its corresponding point in the second point cloud.
3. Transform the points of the first point cloud using the estimated combination of rotation and translation parameters.
4. Iterate steps 1-3 using the transformed points of the first point cloud.
   In this example, after performing an appropriate number of iterations, registration system 716 may determine rotation and translation parameters that describe a spatial relationship between the original positions of the points in the first point cloud and the final positions of the points in the first point cloud. The determined rotation and translation parameters can therefore express a mapping between the first point cloud and the second point cloud. Registration data 710 may include the determined rotation and translation parameters. In this way, registration system 716 may generate registration data 710.

Plan data 712 may include data information describing a plan for a clinician to follow with respect to a patient. In some examples, plan data 712 may include surgical planning data that describe a process to prepare for and conduct a surgery on the patient. For instance, plan data 712 may include data defining a modified surface of a target bone to be used as a bone graft. In some examples, plan data 712 may include other information describing a process to prepare for and conduct the surgery on the patient. For instance, plan data 712 may include information defining a planned reaming axis and position for reaming the patient's scapula, information defining a planned axis for inserting a surgical pin in a humerus for extracting a bone fragment to use as the target bone, and other details of the surgery. In some examples, plan data 712 also includes medical images, e.g., x-ray images, computed tomography images or models, and so on.

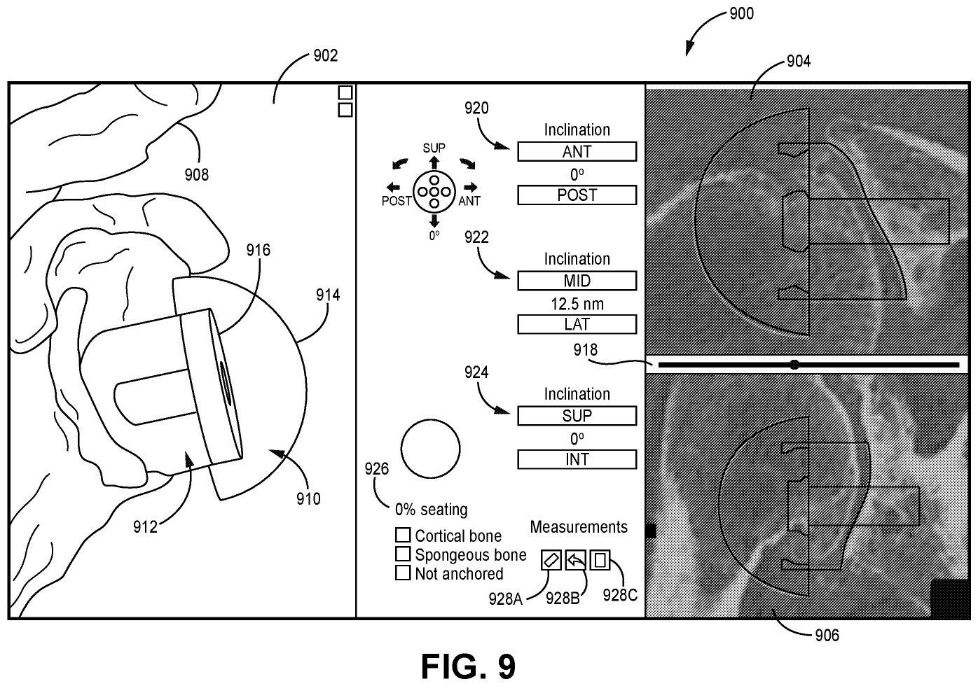

Planning system 718 may enable a user to view plan data 712. For instance, planning system 718 may cause a display device (e.g., MR. visualization device 104, display 708, etc.) to output one or more graphical user interfaces that enable the user to see models of anatomic structures, prostheses, bone grafts, and so on. In some examples, planning system 718 may generate some or all of plan data 712 in response to input from the user. For example, planning system 718 may generate, based on user input, data defining a surface of a target bone for use as a bone graft. An example of generating a surface is described below with respect to FIG. 9. In some examples, planning system 718 predicts a surface of the target bone for use as a bone graft in the patient. In some such examples, planning system 718 applies a machine-learned model (e.g., a neural network, support vector machine, etc.) to predict the surface of the target bone.

Furthermore, virtual guidance system 720 may cause MR visualization device 104 to output virtual objects for display. In accordance with the techniques of this disclosure, while target bone 116 is positioned on bone support member 110 of platform 106 and while a position in the coordinate system of target bone 116 and the position in the coordinate system for virtual object 122 is within a field of view of MR visualization device 104, virtual guidance system 720 may cause MR visualization device 104 to output virtual object 122 for display so that virtual object 122 appears to a user of MR visualization device 104 to pass through target bone 116 and indicates the planned surface of target bone 116.

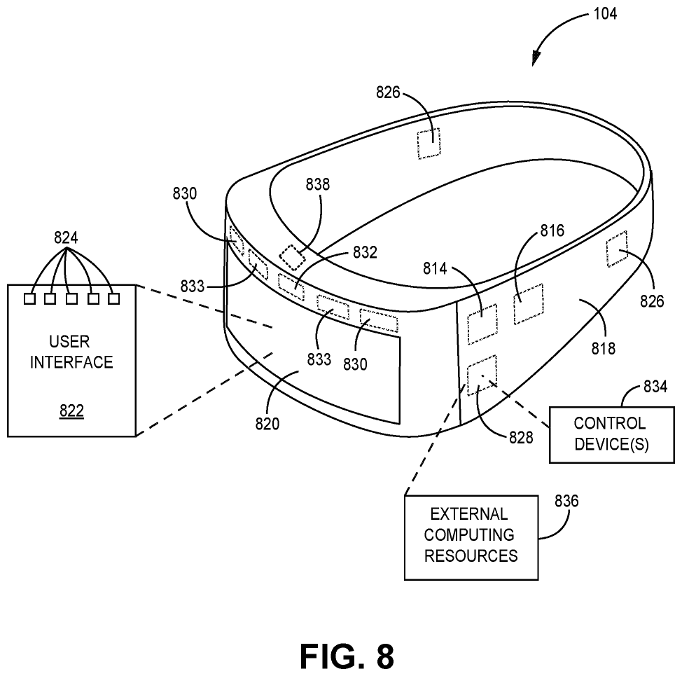

FIG. 8 is a schematic representation of MR visualization device 104 in accordance with one or more techniques of this disclosure. As shown in the example of FIG. 8, MR visualization device 104 can include a variety of electronic components found in a computing system, including one or more processor(s) 814 (e.g., microprocessors or other types of processing units) and memory 816 that may be mounted on or within a frame 818. In some examples, processing circuitry 702 may include processors 814 and/or memory 704 may include memory 816.

Furthermore, in the example of FIG. 8, MR visualization device 104 may include a transparent screen 820 that is positioned at eye level when MR visualization device 104 is worn by a user. In some examples, screen 820 can include one or more liquid crystal displays (LCDs) or other types of display screens on which images are perceptible to a user who is wearing or otherwise using MR visualization device 104 via screen 820. Other display examples include organic light emitting diode (OLED) displays. In some examples, MR visualization device 104 can operate to project 3D images onto the user's retinas using techniques known in the art.

In some examples, screen 820 includes see-through holographic lenses, sometimes referred to as waveguides, that permit a user to see real-world objects through (e.g., beyond) the lenses and also see holographic imagery projected into the lenses and onto the user's retinas by displays, such as liquid crystal on silicon (LCoS) display devices, which are sometimes referred to as light engines or projectors, operating as an example of a holographic projection system 838 within MR visualization device 104. In other words, MR visualization device 104 may include one or more see-through holographic lenses to present virtual images to a user. Hence, in some examples, MR visualization device 104 can operate to project 3D images onto the user's retinas via screen 820, e.g., formed by holographic lenses. In this manner, MR visualization device 104 may be configured to present a 3D virtual image to a user within a real-world view observed through screen 820, e.g., such that the virtual image appears to form part of the real-world environment. In some examples, MR visualization device 104 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Although the example of FIG. 8 illustrates MR visualization device 104 as a head-wearable device, MR visualization device 104 may have other forms and form factors. For instance, in some examples, MR visualization device 104 may be a handheld smartphone or tablet. In other examples, MR visualization device 104 is a supported by an armature that allows the clinician to move MR visualization device 104 into and out of a position for viewing target bone 116 without the clinician wearing MR visualization device 104.

MR visualization device 104 can also generate a user interface (UI) 822 that is visible to the user, e.g., as holographic imagery projected into see-through holographic lenses as described above. For example, UI 822 can include a variety of selectable widgets 824 that allow the user to interact with a MR system. Imagery presented by MR visualization device 104 may include, for example, one or more 2D or 3D virtual objects. MR visualization device 104 also can include a speaker or other sensory devices 826 that may be positioned adjacent the user's ears. Sensory devices 826 can convey audible information or other perceptible information (e.g., vibrations) to assist the user of MR visualization device 104.

MR visualization device 104 can also include a transceiver 828 to connect MR visualization device 104 to a network, a computing cloud, such as via a wired communication protocol or a wireless protocol, e.g., Wi-Fi, Bluetooth, etc. MR visualization device 104 also includes a variety of sensors to collect sensor data, such as one or more optical sensor(s) 830 and one or more depth sensor(s) 832 (or other depth sensors), mounted to, on or within frame 818. In some examples, optical sensor(s) 830 are operable to scan the geometry of the physical environment in which a user of MR visualization device 104 is located (e.g., an operating room) and collect two-dimensional (2D) optical image data (either monochrome or color). Depth sensor(s) 832 are operable to provide 3D image data, such as by employing time of flight, stereo or other known or future-developed techniques for determining depth and thereby generating image data in three dimensions. Other sensors can include motion sensors 833 (e.g., Inertial Mass Unit (IMU) sensors, accelerometers, etc.) to assist with tracking movement.

Computing system 102 may receive tracking data from sensors of MR visualization device 104. The tracking data may include data from optical sensors 830, depth sensors 832, motion sensors 833, and so on. Computing system 102 may process the tracking data so that geometric, environmental, textural, or other types of landmarks (e.g., corners, edges or other lines, walls, floors, objects) in the user's environment or “scene” can be defined and movements within the scene can be detected. As an example, the various types of tracking data can be combined or fused so that the user of MR visualization device 104 can perceive virtual objects that can be positioned, or fixed and/or moved within the scene. When a virtual object is fixed in the scene, the user can walk around the virtual object, view the virtual object from different perspectives, and manipulate the virtual object within the scene using hand gestures, voice commands, gaze line (or direction) and/or other control inputs. In some examples, computing system 102 may process the tracking data so that the user can position a 3D virtual object (e.g., a virtual object indicating a planned surface of target bone 116) on an observed physical object in the scene (e.g., target bone 116) and/or orient the 3D virtual object with other virtual objects displayed in the scene. In some examples, computing system 102 may process the tracking data so that the user can position and fix a virtual representation of the surgical plan (or other widget, image or information) onto a surface, such as a wall of the operating room. In some examples, computing system 102 may use the tracking data to recognize surgical instruments and determine the positions of those surgical instruments.

MR visualization device 104 may include one or more processors 814 and memory 816, e.g., within frame 818 of MR visualization device 104. In some examples, one or more external computing resources 836 process and store information, such as sensor data, instead of or in addition to in-frame processor(s) 814 and memory 816. For example, external computing resources 836 may include processing circuitry, memory. and/or other computing resources of computing system 102 (FIG. 1), In this way, data processing and storage may be performed by one or more processors 814 and memory 816 within MR visualization device 104 and/or some of the processing and storage requirements may be offloaded from MR visualization device 104. Hence, in some examples, one or more processors that control the operation of MR visualization device 104 may be within MR visualization device 104, e.g., as processor(s) 814. Alternatively, in some examples, at least one of the processors that controls the operation of MR visualization device 104 may be external to MR visualization device 104, e.g., as processor(s) 814. Likewise, operation of MR visualization device 104 may, in some examples, be controlled in part by a combination of one or more processors 814 within the visualization device and one or more processors external to MR visualization device 104.

In some examples, processing of tracking data can be performed by processor(s) 814 in conjunction with memory 816 or memory 704. In some examples, processor(s) 814 and memory 816 mounted to frame 818 may provide sufficient computing resources to process the tracking data collected by optical sensor(s) 830, depth sensor(s) 832 and motion sensors 833. In some examples, the tracking data can be processed using a Simultaneous Localization and Mapping (SLAM) algorithm, or other algorithms for processing and mapping 2D and 3D image data and tracking the position of MR. visualization device 104 in the 3D scene. In some examples, image tracking may be performed using sensor processing and tracking functionality provided by the Microsoft HOLOLENS™ system, e.g., by one or more sensors and processors 814 within a MR visualization device 104 substantially conforming to the Microsoft HOLOLENS™ device or a similar mixed reality (MR) visualization device.

In some examples, system 100 can also include user-operated control device(s) 834 that allow the user to operate MR visualization device 104, use MR visualization device 104 in spectator mode (either as master or observer), interact with UI 822 and/or otherwise provide commands or requests to processors(s) 814 or other systems connected to a network. As examples, control device(s) 834 can include a microphone, a touch pad, a control panel, a motion sensor or other types of control input devices with which the user can interact.

FIG. 9 is a conceptual diagram illustrating an example user interface (UI) 900 of planning system 718 for a bone graft, according to techniques of this disclosure. Planning system 718 may output UI 900 for display (e.g., on display 708) while a user (e.g., a clinician, surgeon, etc.) is planning a shoulder arthroplasty. UI 900 includes a 3-dimensional (3D) model view 902, a 2-dimensional (2D) superior view 904, and a 2D anterior view 906. 3D model view 902 includes a 3D bone model 908, a 3D prosthesis model 910, and a 3D bone graft model 912. In the example of FIG. 9, bone model 908 is a model of the patient's scapula. In other examples, the techniques of this disclosure may be applied to other bones of the patient's body.

Prosthesis model 910 includes a glenosphere model 914 and a baseplate model 916. Glenosphere model 914 is a model of a glenosphere. A glenosphere is a hemispherical structure that provides an articulating surface that mates with a corresponding articulating surface of a concave humeral prosthesis. Baseplate model 916 is a model of a baseplate. A clinician may pass fixation members (e.g., screws) through the baseplate, through the bone graft, and into the patient's scapula to attach the baseplate to the patient's scapula. The glenosphere is connected to the baseplate after the baseplate is attached to the patient's scapula. In the example of FIG. 9, the baseplate includes a central peg that extends into the patient's scapula.

Superior view 904 shows a tomographic slice of a patient's shoulder joint from above (superior to) the shoulder joint looking in a downward (inferior) direction. Anterior view 906 shows a tomographic slice of the patient's shoulder joint from the anterior of the patient looking in a posterior direction. Superimposed in superior view 904 and anterior view 906 are outlines of prosthesis model 910 and bone graft model 912. Planning system 718 changes the tomographic slice shown in superior view 904 in response to user input to adjust a slider control 918. A similar slider control, which is not shown in the example of FIG. 9, may be used to change which tomographic slice is shown in anterior view 906.

Furthermore, UI 900 includes controls for adjusting positioning aspects of the prosthesis. For instance, UI 900 include controls 920 for changing a version angle of the prosthesis in anterior and posterior directions, controls 922 for changing lateralization of the prosthesis in medial and lateral directions, and controls 924 for changing an inclination angle of the prosthesis in superior and inferior directions.

As shown in 3D model view 902, superior view 904, and anterior view 906, bone graft model 912 occupies a space between baseplate model 916 and the patient's scapula. As a user uses controls 918 to change the version angle, lateralization, and inclination angle of the prosthesis, planning system 718 may update the shape of bone graft model 912 so that bone graft model 912 continues to occupy the space between baseplate model 916 and the patient's scapula. For instance, if the user uses controls 922 to increase the lateralization of the prosthesis, planning system 718 may update the shape of bone graft model 912 to increase the lateral thickness of bone graft model 912. Similarly, if the user uses controls 920 to increase the version angle of the prosthesis in the anterior direction, planning system 718 may update the shape of bone graft model 912 to decrease a volume of bone graft model 912 at the anterior side of bone graft model 912, while retaining a lateral surface of bone graft model 912 flush with baseplate model 916 and a medial surface of bone graft model 912 flush with the patient's scapula.

UI 900 also includes a seating indicator 926. Seating indicator 926 indicates a ratio between the bone cover and the support of the baseplate above. If the ratio is 100%, the bone supports the entire surface of the plate. In some examples, a minimum of 80% is needed to be able to place the implant on the patient.

Furthermore, in the example of FIG. 9, UI 900 includes measurement controls 928A, 928B, and 928C (collectively, “measurement controls 928”). When planning system 718 receives an indication of user input to select measurement control 928A. planning system 718 activates a model in which planning system 718 receives indications of user input indicating two locations in 3D model view 902, superior view 904, or anterior view 906. In response, planning system 718 may indicate a distance between the locations. For instance, planning system 718 may indicate that the distance between the locations is 1.5 centimeters (cm). Planning system 718 undoes the placement of a location in response to receiving an indication of user selection of control 928B. Planning system 718 discards (e.g., hides) an indication of a measurement in response to receiving an indication of user selection of control 928C.

After the user has finished planning the placement of the prosthesis, planning system 718 may use bone graft model 912 to define a modified surface of a target bone to be used as the bone graft. For example, planning system 718 may define the modified surface to exactly match the contours of the medial surface of bone graft model 912. In some examples, planning system 718 may define the modified surface as a simplified version of the medial surface of bone graft model 912. The simplified version of the medial surface of bone graft model 912 is not necessarily an exact match to the contours of patient's scapula, but it may be easier for a clinician to shape a target bone to have the simplified rather than the exact version of the medial surface of bone graft model 912. In some examples, planning system 718 may determine the simplified version of the medial surface of bone graft model 912 by identifying a limited number of planes (e.g., 2, 3, 4, etc.) that minimize a difference measure (e.g., sum of absolute differences) between points on the planes and corresponding points on the patient's scapula subject to one or more constraints (e.g., a portion of a plane defining a portion of the bone graft cannot pass through a portion of the patient's scapula).

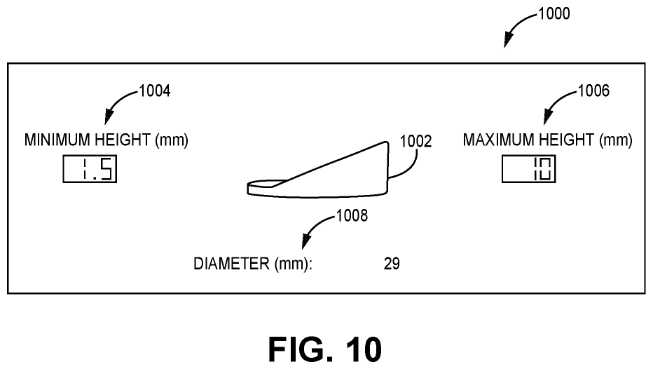

FIG. 10 is a conceptual diagram illustrating an example UI 1000 providing details of a planned bone graft, according to techniques of this disclosure. UI 1000 includes a bone graft model 1002. UI 1000 also includes a minimum height element 1004 indicating a height of the bone graft at its minimum height. In the example of FIG. 10, the minimum height of the bone graft is 1.5 millimeters (mm) and is shown at the left side of bone graft model 1002. UI 1000 also includes a maximum height element 1006 indicating a height of the bone graft at its maximum height. In the example of FIG. 10, the maximum height of the bone graft is 10 mm and is shown at the right side of bone graft model 1002. Additionally, UI 1000 indicates a diameter 1008 of the bone graft. In the example of FIG. 10, the diameter of the bone graft is 29 mm. In other examples, the bone graft has other diameters, such as 25 mm, 27 mm, etc. The bone graft can have various widths, where width is orthogonal to a radius of the bone graft. For example, the bone graft may have widths such as 10 mm, 15 mm, 22 mm, etc.

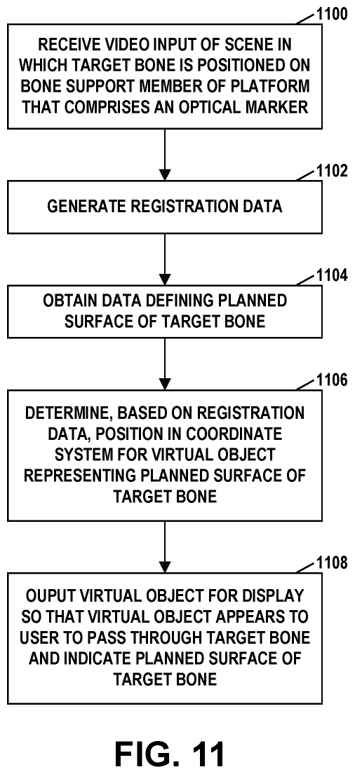

FIG. 11 is a flowchart illustrating an example operation of computing system 102 in accordance with one or more techniques of this disclosure. FIG. Il is described with reference to the examples in other figures of this disclosure. However, the operation of FIG. 11 is not necessarily limited to these examples.

In the example of FIG. 11, computing system 102 receives tracking input of a scene in which a target bone 116 is positioned on a bone support member 110 of a platform 106 that comprises a reference marker 112 (1100). In some examples, optical sensor(s) 830, depth sensor(s) 832, or another sensor of MR visualization device 104 generates the tracking input. In some examples, one or more sensors external to MR visualization device 104 generate the tracking input. For example, a tracking system may include one or more sensors (e.g., cameras, depth sensors, etc.) positioned in a room where platform 106 is being used. The tracking system may track the position of MR visualization device 104 as well as the position of platform 106. In some such examples, reference markers may be attached to MR visualization device 104 to ease tracking of MR visualization device.

In some examples, target bone 116 is cylindrical and bone support member 110 is cylindrical and has a raised rim 300 (FIG. 3) that has an inner diameter that substantially matches an outer diameter of target bone 116. Furthermore, in some examples, platform 106 comprises base plate 108 and a marker stem 114 that supports reference marker 112 at a predefined height above base plate 108. Bone support member 110 and marker stem 114 are connected to base plate 108.

Furthermore, in the example of FIG. 11, computing system 102 may generate registration data 710 that registers reference marker 112 with a coordinate system (1102). This disclosure may refer to the coordinate system as a “real world” coordinate system. Computing system 102 may generate registration data 710 as described elsewhere in this disclosure, e.g., with respect to FIG. 7.

Computing system 102 may obtain data defining a planned surface of target bone 116 (1104). For example, computing system 102 may generate the data defining the planned surface of the target bone, e.g., as described above with respect to the example of FIG. 9. In some examples, computing system 102 may receive the data defining the planned surface of target bone 116, e.g., from a data storage medium, from a software system operating outside of computing system 102, or from other sources. The planned surface of target bone 116 is shaped to engage a second bone (e.g., scapula) of the patient. Thus, in some such examples, the planned surface of target bone 116 is a first surface of target bone 116 and a second surface of target bone 116 opposite the first surface of target bone 116 is shaped to engage an orthopedic prosthesis.

Computing system 102 may determine, based on registration data 710, a position in the coordinate system for a virtual object 122 representing the planned surface of target bone 116 (1106). Virtual object 122 may indicate one or more cutting planes. As described elsewhere in this disclosure, registration data 710 may define transforms between a virtual coordinate system and the “real world” coordinate system. Virtual object 122 may be defined in terms of the virtual coordinate system. For instance, vertices or other locations in virtual object 122 may be defined in terms of locations in the virtual coordinate system. However, for MR visualization device 104 to display virtual object 122 at an appropriate location with respect to objects in the real world, computing system 102 may use registration data 710 to convert the coordinates in the virtual coordinate system into coordinates in the “real world” coordinate system. In this way, computing system 102 may determine, based on registration data 710, a position in the coordinate system (i.e., the “real world” coordinate system”) for virtual object 122 representing the planned surface of target bone 116.

While target bone 116 is positioned on bone support member 110 of platform 106 and while a position in the coordinate system of target bone 116 and the position in the coordinate system for virtual object 122 is within a field of view of MR visualization device 104, computing system 102 may cause MR visualization device 104 to output virtual object 122 for display so that virtual object 122 appears to a user of MR visualization device 104 to pass through target bone 116 and indicates the planned surface of target bone 116 (1108). For example, one or more computing devices of computing system 102 may send instructions to MR visualization device 104 that instruct MR visualization device 104 to output virtual object 122 for display. In some examples, MR visualization device 104 is otherwise preconfigured to display virtual object 122 when the position in the coordinate system for virtual object 122 is within a field of view of MR visualization device 104. Thus, when a clinician using MR visualization device 104 looks at target bone 116, the clinician sees virtual object 122 overlaid on target bone 116. The clinician may therefore use virtual object 122 as a guide for shaping target bone 116.

In some examples, computing system 102 tracks a position of a surgical instrument (e.g., an oscillating saw, cutting burr, etc.) as a clinician uses the surgical instrument to shape target bone 116. In some such examples, a reference marker is attached to the surgical instrument to case tracking of the surgical instrument. Computing system 102 may provide feedback to the clinician as the clinician uses the surgical tool to shape target bone 116 according to the planned surface. In other words, computing system 102 may provide feedback to the user of MR visualization device 104 based on alignment of a surgical instrument with the planned surface of target bone 116. For instance, computing system 102 may change colors of virtual object 122 when the surgical instrument is or is not in alignment with the planned surface. In some examples, computing system 102 may provide audible or tactile feedback to the clinician based on whether the surgical instrument is or is not in alignment with the planned surface. In some examples, computing system 102 may cause sensory devices 826 of MR visualization device 104 to output audible feedback.

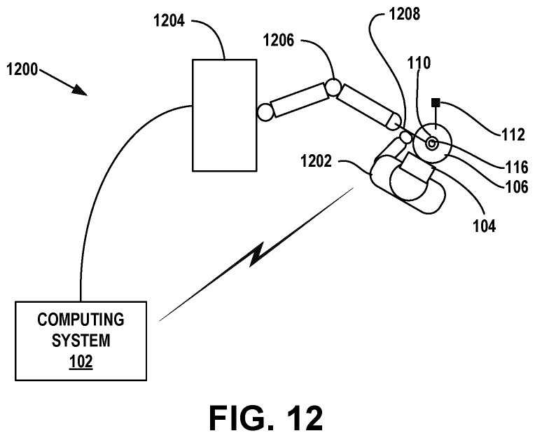

FIG. 12 is a conceptual diagram illustrating an example system 1200 in which a clinician 1202 uses robotic assistance to shape a target bone in accordance with one or more techniques of this disclosure. Clinician 1202 does not form part of system 1200. In the example of FIG. 12, system 1200 includes a robot 1204 having a robotic arm 1206. A surgical instrument 1208 (e.g., an oscillating saw, cutting burr, etc.) is connected to robotic arm 1206. When shaping target bone 116, clinician 1202 may hold and maneuver surgical instrument 1208. Robotic arm 1206 may stabilize surgical instrument 1208 as clinician 1202 maneuvers surgical instrument 1208. By stabilizing surgical instrument 1208, robotic arm 1206 may prevent damage to target bone 116 when clinician 1202 is shaping target bone 116, e.g., due to the clinician's grip on surgical instrument 1208 slipping, unsteadiness of the clinician's hand, clinician 1202 not following a planned surface when shaping target bone 116, and so on.

In the example of FIG. 12, while target bone 116 is positioned on bone support member 110 of platform 106 and while a position in the coordinate system of target bone 116 and the position in the coordinate system for a virtual object is within a field of view of MR visualization device 104, computing system 102 may cause MR visualization device 104 to output a virtual object (not shown in FIG. 12) for display so that the virtual object appears to clinician 1202 to pass through target bone 116 and indicates the planned surface of target bone 116. In some examples, computing system 102 may use tracking data to determine a position of robotic arm 1206 in the “real world”coordinate system.

The following is a non-limiting list of clauses in accordance with techniques of this disclosure.
Clause 1. A method comprising: receiving, by a computing system, tracking input of a scene in which a target bone is positioned on a bone support member of a platform, wherein the platform comprises a reference marker; generating, by the computing system, registration data that registers the reference marker with a coordinate 24 system; obtaining, by the computing system, data defining a planned surface of the target bone; determining, by the computing system, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of a mixed reality (MR) visualization device, causing, by the computing system, the MR visualization device to output the virtual object for display so that the virtual object appears to a user of the MR visualization device to pass through the target bone and indicates the planned surface of the target bone.

Clause 2. The method of clause 1, wherein the virtual object indicates one or more cutting planes.

Clause 3. The method of any of clauses 1-2, wherein the planned surface of the target bone is shaped to engage a second bone of a patient.

Clause 4. The method of any of clauses 1-3, wherein: the planned surface of the target bone is a first surface of the target bone, and a second surface of the target bone opposite the first surface of the target bone is shaped to engage an orthopedic prosthesis.

Clause 5. The method of any of clauses 1-4, wherein: the target bone is cylindrical, and the bone support member is cylindrical and has a raised rim that has an inner diameter that substantially matches an outer diameter of the target bone.

Clause 6. The method of any of clauses 1-5, wherein: the platform further comprises: a base plate; and a marker stem that supports the reference marker at a predefined height above the base plate, wherein the bone support member and the marker stem are connected to the base plate.

Clause 7. The method of any of clauses 1-6, further comprising providing, by the computing system, feedback to the user of the MR visualization device based on alignment of a surgical instrument with the planned surface of the target bone.

Clause 8. A system comprising: a mixed reality (MR) visualization device; and processing circuitry configured to: receive tracking input of a scene in which a target bone is positioned on a bone support member of a platform, wherein the platform comprises a reference marker; generate registration data that registers the reference marker with a coordinate system; obtain data defining a planned surface of the target bone; determine, based on the registration data, a position in the coordinate system for a virtual object representing the planned surface of the target bone; and while the target bone is positioned on the bone support member of the platform and while a position in the coordinate system of the target bone and the position in the coordinate system for the virtual object is within a field of view of the MR visualization device, cause the MR visualization device to output the virtual object for display so that the virtual object appears to a user of the MR visualization device to pass through the target bone and indicates the planned surface of the target bone.

Clause 9. The system of clause 8, wherein the virtual object indicates one or more cutting planes.

Clause 10. The system of any of clauses 8-9, wherein the planned surface of the target bone is shaped to engage a second bone of a patient.

Clause 11. The system of clause 10, wherein: the planned surface of the target bone is a first surface of the target bone, and a second surface of the target bone opposite the first surface of the target bone is shaped to engage an orthopedic prosthesis.

Clause 12. The system of any of clauses 8-11, wherein: the target bone is cylindrical, and the bone support member is cylindrical and has a raised rim that has an inner diameter that substantially matches an outer diameter of the target bone.

Clause 13. The system of clause 12, wherein the bone support member has a raised central protrusion having a diameter that approximately matches a diameter of a central circular incision in the target bone.

Clause 14. The system of any of clauses 8-13, wherein: the platform further comprises: a base plate; and a marker stem that supports the reference marker at a predefined height above the base plate, wherein the bone support member and the marker stem are connected the base plate.

Clause 15. The system of any of clauses 8-14, wherein the reference marker is a cube having different predefined optical patterns on each face other than a face to which the marker stem is connected.

Clause 16. The system of any of clauses 8-14, wherein the processing circuitry is further configured to provide feedback to the user of the MR visualization device based on alignment of a surgical instrument with the planned surface of the target bone.

Clause 17. The system of any of clauses 8-16, further comprising a robot having a robotic arm configured to stabilize a surgical instrument used to shape the target bone.

Clause 18. A non-transitory computer-readable storage medium having instructions stored thereon that, when executed, cause a computing system to perform the methods of any of clauses 1-7.

Clause 19. A system comprising means for performing the methods of any of clauses 1-7.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuity,” as used herein may refer to any of the foregoing structures or any other structure suitable for implementation of the techniques described herein,

1. A method comprising:
receiving, by a computing system, tracking input of a scene in which a target bone is positioned on a bone support member of a platform, wherein the platform comprises a reference marker;
generating, by the computing system, registration data that registers the reference marker with a coordinate system;
obtaining, by the computing system, data defining a planned surface of the target bone;
determining, by the computing system, based on the registration data, a position in the coordinate system for a virtual object representing the planned surface of the target bone; and
while the target bone is positioned on the bone support member of the platform and while a position in the coordinate system of the target bone and the position in the coordinate system for the virtual object is within a field of view of a mixed reality (MR) visualization device, causing, by the computing system, the MR visualization device to output the virtual object for display so that the virtual object appears to a user of the MR visualization device to pass through the target bone and indicates the planned surface of the target bone.

2. The method of claim 1, wherein the virtual object indicates one or more cutting planes.

3. The method of claim 1, wherein the planned surface of the target bone is shaped to engage a second bone of a patient.

4. The method of claim 1, wherein:
the planned surface of the target bone is a first surface of the target bone, and
a second surface of the target bone opposite the first surface of the target bone is shaped to engage an orthopedic prosthesis.

5. The method of claim 1, wherein:
the target bone is cylindrical, and
the bone support member is cylindrical and has a raised rim that has an inner diameter that substantially matches an outer diameter of the target bone.

6. The method of claim 1, wherein:
the platform further comprises:
a base plate; and
a marker stem that supports the reference marker at a predefined height above the base plate, wherein the bone support member and the marker stem are connected to the base plate.

7. The method of claim 1, further comprising providing, by the computing system, feedback to the user of the MR visualization device based on alignment of a surgical instrument with the planned surface of the target bone.

8. A system comprising:
a mixed reality (MR) visualization device; and
processing circuitry configured to:
receive tracking input of a scene in which a target bone is positioned on a bone support member of a platform, wherein the platform comprises a reference marker;
generate registration data that registers the reference marker with a coordinate system;
obtain data defining a planned surface of the target bone;
determine, based on the registration data, a position in the coordinate system for a virtual object representing the planned surface of the target bone; and
while the target bone is positioned on the bone support member of the platform and while a position in the coordinate system of the target bone and the position in the coordinate system for the virtual object is within a field of view of the MR visualization device, cause the MR visualization device to output the virtual object for display so that the virtual object appears to a user of the MR visualization device to pass through the target bone and indicates the planned surface of the target bone.

9. The system of claim 8, wherein the virtual object indicates one or more cutting planes.

10. The system of claim 8, wherein the planned surface of the target bone is shaped to engage a second bone of a patient.

11. The system of claim 10, wherein:
the planned surface of the target bone is a first surface of the target bone, and
a second surface of the target bone opposite the first surface of the target bone is shaped to engage an orthopedic prosthesis.

12. The system of claim 8, wherein:
the target bone is cylindrical, and
the bone support member is cylindrical and has a raised rim that has an inner diameter that substantially matches an outer diameter of the target bone.

13. The system of claim 12, wherein the bone support member has a raised central protrusion having a diameter that approximately matches a diameter of a central circular incision in the target bone.

14. The system of claim 8, wherein:
the platform further comprises:
a base plate; and
a marker stem that supports the reference marker at a predefined height above the base plate, wherein the bone support member and the marker stem are connected the base plate.

15. The system of claim 14, wherein the reference marker is a cube having different predefined optical patterns on each face other than a face to which the marker stem is connected.

16. The system of claim 8, wherein the processing circuitry is further configured to provide feedback to the user of the MR visualization device based on alignment of a surgical instrument with the planned surface of the target bone.

17. The system of claim 8, further comprising a robot having a robotic arm configured to stabilize a surgical instrument used to shape the target bone.

18. One or more non-transitory computer-readable storage media having instructions stored thereon that, when executed by one or more processors of a computing system, cause the computing system to:
receive tracking input of a scene in which a target bone is positioned on a bone support member of a platform, wherein the platform comprises a reference marker;
generate registration data that registers the reference marker with a coordinate system;
obtain data defining a planned surface of the target bone;
determine, based on the registration data, a position in the coordinate system for a virtual object representing the planned surface of the target bone; and
while the target bone is positioned on the bone support member of the platform and while a position in the coordinate system of the target bone and the position in the coordinate system for the virtual object is within a field of view of a mixed reality (MR) visualization device, cause the MR visualization device to output the virtual object for display so that the virtual object appears to a user of the MR visualization device to pass through the target bone and indicates the planned surface of the target bone.

19. (canceled)

20. The one or more non-transitory computer-readable storage media of claim 18, wherein:
the platform further comprises:
a base plate; and
a marker stem that supports the reference marker at a predefined height above the base plate, wherein the bone support member and the marker stem are connected to the base plate.

21. The one or more non-transitory computer-readable storage media of claim 18, wherein the virtual object indicates one or more cutting planes.
